(19) United States
(12) Patent Application Publication
(10) Pub. No.: US 2014/0363900 A1
(43) Pub. Date: Dec. 11, 2014

(54) Giant Porphyrin-Phospholipid Vesicles

(71) Applicant: UNIVERSITY HEALTH NETWORK, Toronto (CA)

(72) Inventors: Gang Zheng, Toronto (CA); Jonathan Lovell, Niagra Falls (CA); Elizabeth Huynh, Mississiauga (CA)

(21) Appl. No.: 14/363,778

(22) PCT Filed: Dec. 5, 2012
(86) PCT No.: PCT/CA2012/001122

§ 371 Date: Jun. 6, 2014

Related U.S. Application Data
(60) Provisional application No. 61/568,352, filed on Dec. 8, 2011.

(51) Int. Cl. C12Q 1/68; G01N 1/28

(52) U.S. Cl. CPC C12Q 1/6806; G01N 1/28
USPC 436/501; 436/94

(57) ABSTRACT

There is provided herein vesicles comprising a bilayer comprising porphyrin-phospholipid conjugate, wherein the porphyrin-phospholipid conjugate comprises one porphyrin, porphyrin derivative or porphyrin analog covalently attached to a lipid side chain, preferably at the sn-1 or the sn-2 position, of one phospholipids, wherein the vesicle is 1-100 microns in diameter.

### RELATED APPLICATIONS

This application claims priority to U.S. Provisional Patent Application No. 61/568,352 filed Dec. 8, 2011.

### FIELD OF THE INVENTION

This invention relates to the field of porphyrin-phospholipid vesicles and, more preferably, to giant porphyrin-phospholipid vesicles capable of spatially and temporally controlled opening and closing.

### BACKGROUND OF THE INVENTION

Phospholipid-enclosed compartments play a central role in cellular and sub-cellular homeostasis, with the bilayer serving as the general barrier between external and internal biomolecules and chemicals. Putative prebiotic bilayers have been recreated in the context of understanding and mimicking how cells came to control the passage and production of biomolecules¹⁻³. A wide range of protein-based transport systems have evolved in organisms to permit the movement of molecules through bilayers without destroying overall membrane integrity. However, these transport systems are typically specific for certain cargo and are not suitable as general purpose gateways to the interior of natural or synthetic phospholipid-enclosed compartments. Thus, disruptive techniques such as electroporation and heat shock have been developed to permit the passage of biomolecules, such as DNA, through cell membranes^{4,5}. Recently, electroporation was used to fuse giant vesicles for nanoparticle synthesis.⁶ While highly practical for some applications, these methods are not easy to control. The opening and closing of swollen, giant lipid vesicles have been well characterized, but the process is not readily controllable and has traditionally made use of highly viscous solvents that preclude many applications.⁷ More precise control of bilayer permeability has been achieved using novel approaches such as local electroporation, proximal heating of gold nanoparticles and electroinjection.⁸⁻¹⁰

### SUMMARY OF THE INVENTION

In an aspect, there is provided, a vesicle comprising a bilayer comprising porphyrin-phospholipid conjugate, wherein the porphyrin-phospholipid conjugate comprises one porphyrin, porphyrin derivative or porphyrin analog covalently attached to a lipid side chain, preferably at the sn-1 or the sn-2 position, of one phospholipids, wherein the vesicle is 1-100 microns in diameter.

In a further aspect, there is provided a method of preparing vesicles, comprising preparing a solution comprising porphyrin-phospholipid conjugate, wherein the porphyrin-phospholipid conjugate comprises one porphyrin, porphyrin derivative or porphyrin analog covalently attached to a lipid side chain of one phospholipid, preferably at the sn-1 or the sn-2 position; the solution optionally further comprising phospholipids and cholesterol; and dehydrating the solution and subjecting a resulting lipid film to an alternating current. Preferably, the solution is coated onto wires, preferably platinum wires, which deliver the alternating current.

In a further aspect, there is provided a vesicle produced by the methods described herein.

In a further aspect, there is provided the vesicle described herein produced by the method described herein.

In a further aspect, there is provided a method of controlled opening of a vesicle, comprising providing the vesicle descried herein and irradiating the vesicle with a laser or other light source, preferably a xenon or halogen lamp, capable of opening the vesicle. In preferred embodiments, the controlled opening is at a predetermined location on the vesicle bilayer and said location is irradiated with the laser. The controlled opening is further preferably at a predetermined time. In preferred embodiments, the controlled opening is performed under a microscope.

In a further aspect, there is provided a use of the vesicle described herein as a bioreactor.

In a further aspect, there is provided a method of performing a bioreaction between at least two reagents in a vesicle, comprising providing the vesicle described herein having a first reagent encapsulated therein; performing controlled opening of the vesicle according to the method described herein to allow the entry of a second reagent into the interior of the vesicle and optionally allowing the vesicle to self-close; and allowing the bioreaction to occur.

### BRIEF DESCRIPTION OF THE DRAWINGS

Embodiments of the invention may best be understood by referring to the following description and accompanying drawings. In the drawings:

FIG. 1 shows the electroformation of self-quenched giant porphyrin vesicles (GPVs). (a) Experimental setup used for GPV electroformation using a low cost, open source microcontroller and common lab equipment. The circuit diagram is shown in the upper left. One potentiometer adjusted the AC voltage (V mod.) and the other adjusted the frequency (f mod.). The square wave output is indicated in the circle shown between the two platinum wires. The apparatus set up is shown on the right. The left insets show a confocal micrograph and a schematic image of GPVs formed on wire. (b) Fluorescence quenching within the bilayer of GPVs formed with sucrose, or on the platinum wire. Confocal microscope fluorescence settings the same in each image. 10 μm scale bar is shown. (c) A more detailed circuit diagram of the circuit in (a).

FIG. 2 shows GPV opening and self-sealing. (a) GPVs containing 70 molar % pyro-lipid opened and self sealed upon laser irradiation (white dashed circle). Arrows show the GPV opening. 10 micron scale bar is indicated (b) Repeated opening and self-sealing of a GPV. Arrows indicate time of laser pulsing. (c) GPV response to laser irradiation with varying laser power and (d) irradiation diameter using a 200 ms irradiation time. Frequency charts are based on results from 10 separate GPVs irradiations per bar.

FIG. 3 shows an estimation of edge tension in GPV pores. A typical pore opening is shown, plotted as R²In(r) as a function of time, where R is the GPV radius and r is the pore radius. The edge tension during the slow close period, based on the slope of the black line, was 19 fN. Laser irradiation parameters used: 200 ms irradiation time, 2 μm irradiation diameter spot size, 100% laser power.

FIG. 4 shows diffusion of biomolecules into GPVs. (a) Confocal images of GPVs and exogenous fluorophores added to the solution. Laser irradiation (fluence of 40 μJ/μm2) location is indicated with a dashed circle. 10 micron scale bar is shown. (b) Diffusion equilibrium of various fluorophores into GPVs. Arrow indicates time point of GPV opening. (c) Half-times to diffusion equilibrium for various sized fluorophores.

FIG. 5 shows optical gating of size dependent cargo out of GPVs. Different molecular weight fluorophores, carboxyfluorescein (0.4 kDa) and TRITC dextran (155 kDa), were co-encapsulated in GPVs and external fluorophores were removed by washing. (a,b) A GPV was irradiated with a pulse of low laser fluence (laser pulse 1: 2 μJ/μm²) and low molecular weight molecules (carboxyfluorescein) were released; however the larger fluorophores (TRITC dextran) remained trapped inside the GPV. 10 μm scale bar shown. (c, d) A GPV was first irradiated with a pulse of low laser fluence (laser pulse 1) and carboxyfluorescein was released while TRITC dextran remained inside. After 2 minutes, a second pulse of greater laser fluence (laser pulse 2: 20 μJ/μm²) was applied and the large TRITC dextran was released. 10 μm scale bar shown.

FIG. 6 shows the Sequential hybridization control of GPV contents. Fluorescently labeled DNA in the exterior of the GPVs (1, yellow) was permitted to enter the GPVs following laser opening (2). A complementary sequence with a quenching moiety was then added to the external medium (3, black circles) and again the GPV was opened to allow the quenching to occur inside the GPV (4). 10 micron scale bar is shown.

FIG. 7 shows a strategy for selective attachment of enzymes to the interior of the GPV. a) Schematic representation of blocking the external leaflet biotin sites using avidin, followed by addition of an avidin-conjugate, and opening and closing of the GPV to selectively place the avidin conjugate (which could be labeled with a fluorophore or attached to another enzyme) inside the GPV. b) Multiple opening and closing events can evenly distribute the avidin-conjugates of interest inside GPVs. Following exterior blocking, FITC-avidin was placed in the medium and the GPVs were opened and closed multiple times in the order the numbering indicates.

FIG. 8 shows membrane stretching following laser irradiation. 10 micron scale bar is shown.

FIG. 9 shows the dependence of low salt for GPV opening. Frequency of opening events is shown for GPVs following laser irradiation in the given salt concentrations

### DETAILED DESCRIPTION OF THE PREFERRED EMBODIMENTS

Efforts to develop self-contained microreactors have been limited by difficulty in generating membranes that can be robustly and repeatedly opened and closed. Here we demonstrate that porphyrin-phospholipid conjugates electro-assembled into microscale giant porphyrin vesicles which could be readily opened using a focused laser beam in situ. The large openings in the porphyrin bilayer resealed within a minute, allowing for spatial and temporal control of biomolecule diffusion into and out of the vesicles, which was dependent on cargo size. The unique permeability characteristics are proposed to be based on porphyrin-stabilized pore edge tension orders of magnitude smaller than that of conventional phospholipids. The giant vesicles could be opened and closed repeatedly in a controlled manner, permitting sequential DNA hybridization reactions to be performed. A biotin-avidin based strategy was developed to selectively attach enzymes of interest to the interior of the vesicles, demonstrating the potential of giant porphyrin vesicles as versatile microreactors.

In an aspect, there is provided, a vesicle comprising a bilayer comprising porphyrin-phospholipid conjugate, wherein the porphyrin-phospholipid conjugate comprises one porphyrin, porphyrin derivative or porphyrin analog covalently attached to a lipid side chain, preferably at the sn-1 or the sn-2 position, of one phospholipids, wherein the vesicle is 1-100 microns in diameter, preferably 10-50 microns in diameter.

Examples of porphyrin-phospholipid conjugates used in forming vesicles of the application are described in co-owned WO 11/044,671.

In increasing preferability, the vesicle comprises between 15-100 molar %, 20-90 molar %, 30-80 molar %, 40-75 molar %, 50-70 molar %, 60-70 molar % and 65-70 molar % porphyrin-phospholipid conjugate.

In a preferred embodiment, the vesicle comprises about 70 molar % porphyrin-phospholipid conjugate.

In some embodiments, the porphyrin, porphyrin derivative or porphyrin analog in the porphyrin-phospholipid conjugate is selected from the group consisting of hematoporphyrin, protoporphyrin, tetraphenylporphyrin, a pyropheophorbide, a bacteriochlorophyll, chlorophyll a, a benzoporphyrin derivative, a tetrahydroxyphenyl chlorin, a purpurin, a benzochlorin, a naphthochlorins, a verdin, a rhodin, a keto chlorin, an azachlorin, a bacteriochlorin, a tolyporphyrin, a benzobacteriochlorin, an expanded porphyrin and a porphyrin isomer. Preferably, the expanded porphyrin is a texaphyrin, a sapphyrin or a hexaphyrin and the porphyrin isomer is a porphycene, an inverted porphyrin, a phthalocyanine, or a naphthalocyanine.

As used herein, “phospholipid” is a lipid having a hydrophilic head group having a phosphate group and hydrophobic lipid tail.

In some embodiments, the phospholipid in the porphyrin-phospholipid conjugate comprises phosphatidylcholine, phosphatidylethanoloamine, phosphatidylserine or phosphatidylinositol. Preferably, the phospholipid comprises an acyl side chain of 12 to 22 carbons.

In some embodiments, the porphyrin in the porphyrin-phospholipid conjugate is pyropheophorbide-a acid.

In some embodiments, the porphyrin in the porphyrin-phospholipid conjugate is a bacteriochlorophyll derivate.

In some embodiments, the phospholipid in the porphyrin-phospholipid conjugate is 1-Palmitoyl-2-Hydroxy-sn-Glycero-3-Phosphocholine or 1-Stearoyl-2-Hydroxy-sn-Gycero-3-Phosphocholine.

In some embodiments, the porphyrin-phospholipid conjugate is pyro-lipid.

In some embodiments, the porphyrin-phospholipid conjugate is oxy-bacteriochlorophyll-lipid.

In some embodiments, the porphyrin is conjugated to the glycerol group on the phospholipid by a carbon chain linker of 0 to 20 carbons.

In some embodiments, the vesicle is substantially spherical.

In some embodiments, the vescle has an enzyme attached to the inner surface of the bilayer.

In some embodiments, the remainder of the bilayer is comprised substantially of other phospholipid. In preferred embodiment, the other phospholipid is selected from the group consisting of selected from the group consisting of phosphatidylcholines, phosphatidylethanolamines, phosphatidic acid, phosphatidylglycerols and combinations thereof. In further preferred embodiments, the other phospholipid is selected from the group consisting of 1,2-dipalmitoyl-sn-glycero-3-phosphatidic acid (DPPA), 1,2-dipalmitoyl-sn-glycero-3-phosphatidylcholine (DPPC), 1,2-distearoyl-sn-glycero-3-phosphocholine (DSPC), 1,2-dimyristoyl-sn-glycero-3-phosphocholine (DMPC), 1,2-dibehenoyl-sn-glycero-3-phosphocholine (DBPC), 1,2-diarachidoyl-sn-glycero-3-phosphatidylcholine (DAPC), 1,2-dilignoceroyl-sn-glycero-3-phosphatidylcholine(DLgPC), 1,2-dipalmitoyl-sn-glycero-3-[phosphor-rac-(1-glycerol)] (DPPG), L-α-phosphatidylcholine, and combinations thereof. In some preferable embodiments, the vesicle further comprises cholesterol. Preferably, the cholesterol is present in a molar ratio of 3:2 of remainder other phospholipid to cholesterol.

In a further aspect, there is provided a method of preparing vesicles, comprising preparing a solution comprising porphyrin-phospholipid conjugate, wherein the porphyrin-phospholipid conjugate comprises one porphyrin, porphyrin derivative or porphyrin analog covalently attached to a lipid side chain of one phospholipid, preferably at the sn-1 or the sn-2 position; the solution optionally further comprising phospholipids and cholesterol; and dehydrating the solution and subjecting a resulting lipid film to an alternating current. Preferably, the solution is coated onto wires, preferably platinum wires, which deliver the alternating current.

In some embodiments, the solution comprises chloroform as the solvent.

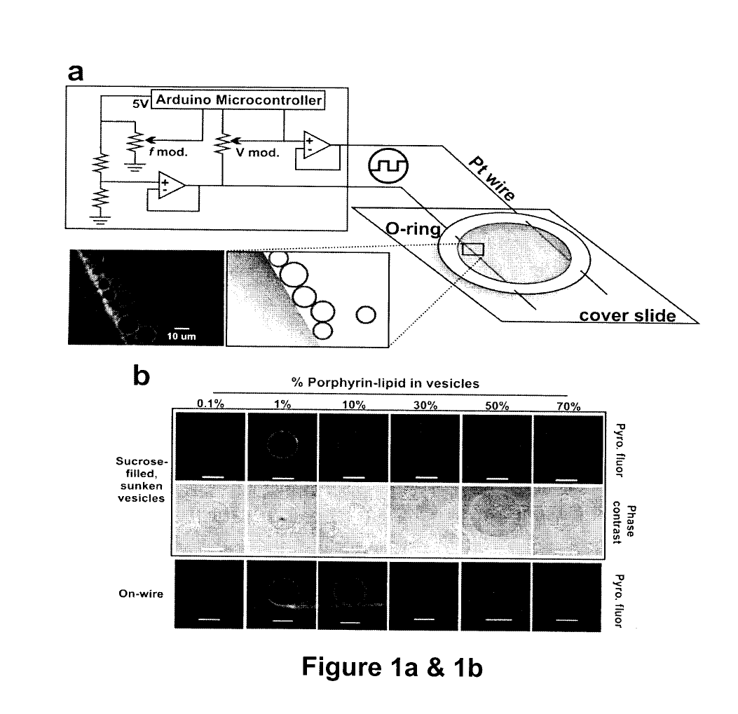

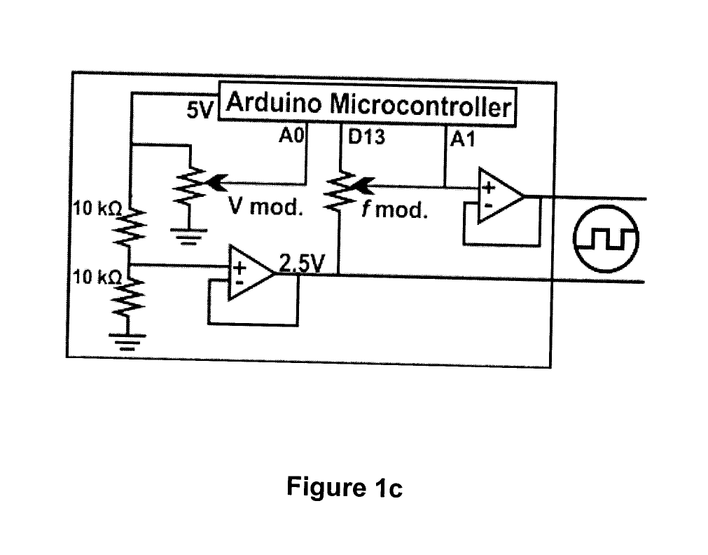

In some embodiments, the alternating current is controlled by an Arduino microcontroller. Preferably the Arduino microcontroller is a part of a circuit as described in FIG. 1a or 1c.

In some embodiments, the method is for preparing the vesicles described herein.

In a further aspect, there is provided a vesicle produced by the methods described herein.

In a further aspect, there is provided the vesicle described herein produced by the method described herein.

In a further aspect, there is provided a method of controlled opening of a vesicle, comprising providing the vesicle descried herein and irradiating the vesicle with a laser or other light source, preferably a xenon or halogen lamp, capable of opening the vesicle. In preferred embodiments, the controlled opening is at a predetermined location on the vesicle bilayer and said location is irradiated with the laser. The controlled opening is further preferably at a predetermined time. In preferred embodiments, the controlled opening is performed under a microscope.

In some embodiments, the laser power is about 660 μW.

In some embodiments, the laser has a wavelength of 405 nm.

In some embodiments, the vesicle is in a solution having a salt concentration of less than 4 mM.

In some embodiments, a size of the opening is controlled proportionally with the level of laser fluence.

In a further aspect, there is provided a use of the vesicle described herein as a bioreactor.

In a further aspect, there is provided a method of performing a bioreaction between at least two reagents in a vesicle, comprising providing the vesicle described herein having a first reagent encapsulated therein; performing controlled opening of the vesicle according to the method described herein to allow the entry of a second reagent into the interior of the vesicle and optionally allowing the vesicle to self-close; and allowing the bioreaction to occur.

The following examples are illustrative of various aspects of the invention, and do not limit the broad aspects of the invention as disclosed herein.

### EXAMPLES

### Methods

All chemical materials were obtained from Sigma and electronic materials were obtained from Mouser, unless indicated otherwise. GPVs were formed using a modified electroformation method.¹² Pyropheophorbide-lipid (prepared as previously described¹¹, but with a modified protocol to generate an isomerically pure conjugate; manuscript submitted) in combination with egg phosphatidylcholine (egg PC) and cholesterol (chol) (3:2 molar ratio egg PC:chol) (Avanti Polar lipids), was dispersed in chloroform to form 0.2 mg/ml-0.5 mg/ml stock solutions. Two 0.5 mm diameter platinum wires (#267228, Sigma) were positioned in parallel separated by a distance of 2 mm through a small polytetrafluoroethylene O-ring (#9559K208, McMaster-Carr) adhered to a cover slide using vacuum grease. 6-10 equally spaced 1 μl droplets of stock solution were deposited on the two platinum wires. Unless otherwise noted 70 molar % pyropheophorbide-lipid was used. Residual chloroform was evaporated by placing the O-ring apparatus in a vacuum for 20 minutes. A 0.6 mL water solution with 2 mM Tris pH 8 was then used to hydrate the lipids on the wire. The apparatus was connected to a 3V, 10 Hz AC current in order to induce electroformation of the vesicles. A low cost, open source Arduino microcontroller was used to generate the field as per the circuit diagram in FIG. 1. Vesicles formed on the wire were visible 15 minutes after turning on the electric field. To visualize vesicles detached from the wire, the lipids were hydrated with a 200 mOsM sucrose solution. After applying the electric field for 45 minutes, 25 μl of the vesicle solution was diluted in 100 μl of a 200 mOsM glucose solution on a cover slide where the vesicles sunk to the bottom of the solution and could be visualized.

Confocal microscopy (Olympus FluoView FV1000) was used to inspect the vesicles using a 633 nm laser and 40× water objective lens. Opening was induced using a 405 nm laser pulse for 200 ms with a power of 660 μW with a 2 μm diameter spot size. For fluorophore diffusion, carboxyfluorescein (81002, AnaSpec Inc.), Texas Red dextran (D-1828, Invitrogen) and TRITC dextran (T1287, Sigma-Aldrich) were added to the medium and observed using a 488 nm laser for carboxyfluorescein and 543 nm for both Texas Red dextran and TRITC dextran. For controlled DNA fluorescence and quenching, an oligonucleotide with the sequence GGTTTTGTTGTTGTTGTTTTC-Fluorescein (Sigma) (SEQ ID NO. 1) was added to the external medium at 1 μM concentration with 1 mM NaCl. After performing light induced loading, the complementary sequence DAB-GAAAACAACAACAACAAAACC (Sigma) (SEQ ID NO. 2) was added to the external medium in a tenfold excess and GPV opening was repeated.

For avidin-biotin binding, 70% porphyrin-lipid GPVs were formed with 0.05% DSPE-Biotin (Avanti Polar Lipids) by depositing eight 1 μl droplets of 0.5 mg/ml on the wires and rehydrating with water. Once formed, 2 mM Tris pH8 was added with 12 nM avidin (AVD407, BioShop Canada Inc.) to the external medium to block the biotin binding sites on the outer leaf of the GPV. After 15 minutes, 24 nM fluorescein conjugated avidin (APA011F, BioShop Canada Inc.) was added to the buffer and the GPV was opened several times to observe fluorescent avidin binding.

### Results

We recently reported that porphyrin-lipid conjugates could self-assemble into liposome-like nanovesicles formed from a porphyrin bilayer.¹¹ To examine whether larger micron-sized porphyrin vesicles could be formed, we developed a modified electroformation approach, based on the alternating current method.¹² Using a low cost, open-source programmable Arduino microcontroller, a solution of varying fractions of porphyrin-lipid and egg phosphatidylcholine with cholesterol in chloroform was coated onto platinum wires, evaporated, rehydrated and subjected to a low-frequency alternating square wave field (FIGS. 1a and 1c). Using this method, micron-scale vesicles were readily generated and could be visualized using confocal microscopy (FIG. 1a, inset). In the presence of the electric field, vesicles formed spontaneously and slowly detached from the platinum wires, and this process continued repeatedly over time. Removal of the electric field prevented the oscillations necessary for the vesicle detachment, leaving a high density of relatively immobilized porphyrin vesicles proximal to the wires, greatly facilitating time-series observation by confocal microscopy. Because the porphyrin component of the lipid conjugate, pyropheophporbide (pyro), is fluorescent, the bilayer could be imaged using fluorescence microscopy without any exogenous label. We examined how increasing proportions of porphyrin-lipid affected two types of movement-restricted vesicles: Detached, sucrose-containing vesicles that sank when transferred to a separate solution of lesser density glucose; and vesicles immobilized on the platinum wire. In both cases, spherical vesicles from 10-50 microns were formed, and addition of greater than 1 molar % porphyrin-lipid led to fluorescence self-quenching of the entire vesicle, despite the higher porphyrin content (FIG. 1b). The consistent porphyrin depth within the bilayer held in place by the amphipathic nature of the porphyrin-lipid, combined with the high porphyrin density suggest that the bilayer environment created dynamic face to face porphyrin interactions, leading to fluorescence quenching. However, microvesicle yield and circular geometry quality decreased beyond 70 molar % porphyrin-lipid, demonstrating that some standard phospholipids were helpful to form the giant porphyrin vesicles (GPVs).

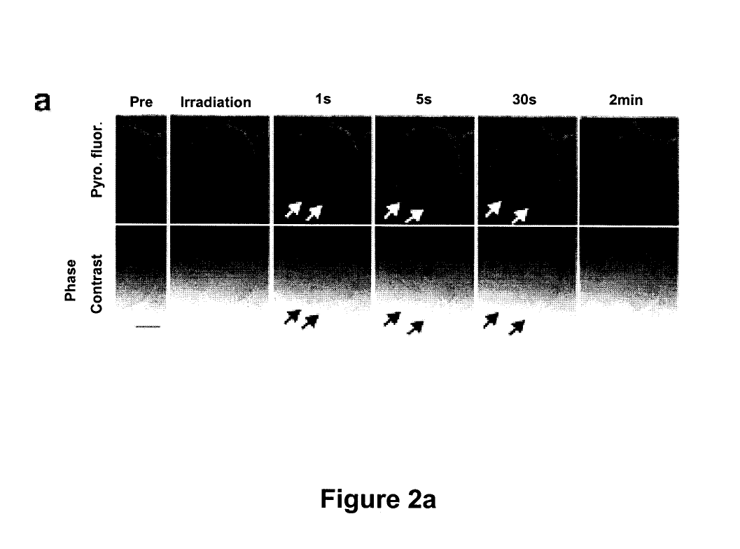

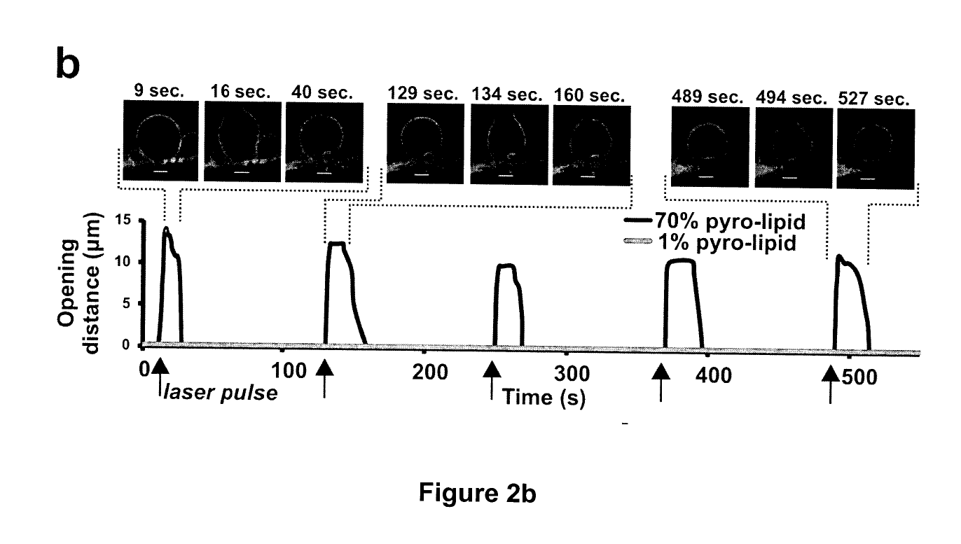

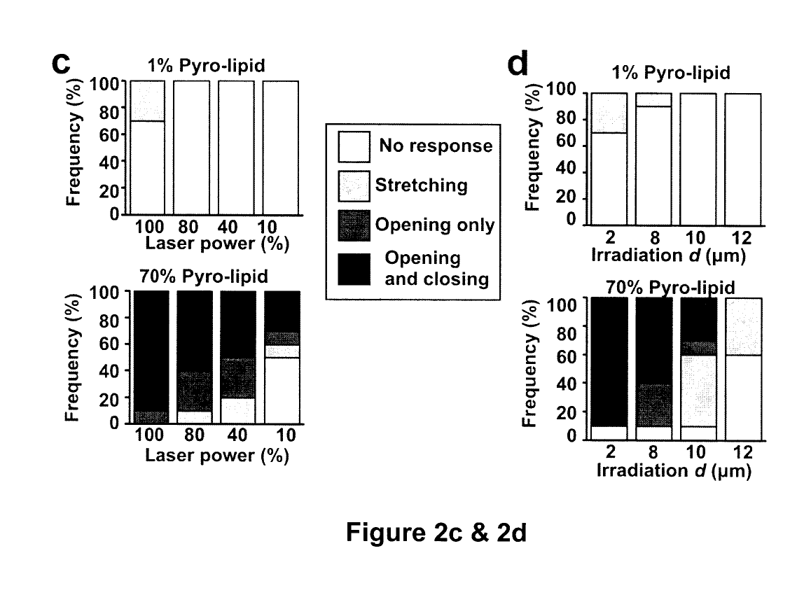

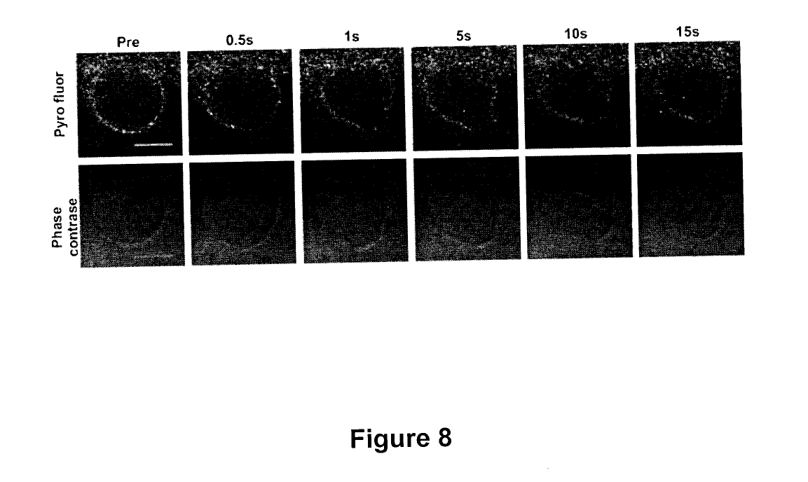

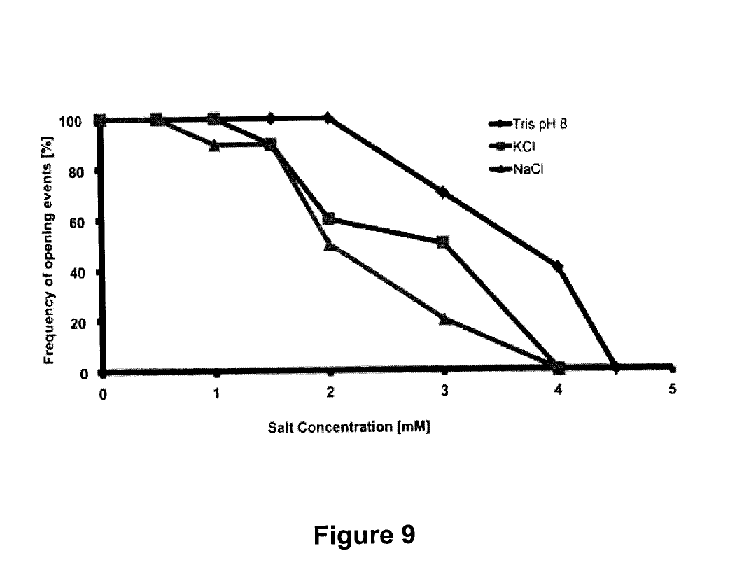

Each 10 micron porphyrin vesicle formed from 70 molar % porphyrin-lipid was estimated to contain approximately 6×10⁸ porphyrins, all confined to the thin, enclosing porphyrin bilayer. Given the high optical absorption of the porphyrin bilayer, the membrane response to laser irradiation was investigated. Despite the high level of fluorescence self-quenching, the bilayer retained enough fluorescence to enable clear optical observation of the bilayer response using a 633 nm laser to excite the Q-band of the porphyrin. The high power laser pulse wavelength was 405 nm, which directly excited the more intense Soret band. The laser power was estimated to be 660 μW, but it was focused into a small volume to achieve power density on the order of kWs per cm². When the bilayer was subjected to a 200 ms pulse, the bilayer was observed to open for an extended period of time (FIG. 2a). After 30 seconds, the open membrane edges came together, resealed and the vesicle appeared intact again. Although they display less contrast, phase contrast images confirmed the bilayer was physically opening and resealing, as opposed to a local fluorescence bleaching of the bilayer. Upon resealing, the vesicle appeared to be completely intact, and therefore, repeated opening and self-sealing of single GPVs was investigated. GPV opening was limited to low salt conditions (FIG. 9). As shown in FIG. 2b, a single GPV could be repeatedly opened and closed indefinitely. In general, the maximum spacing between the two open ends of the GPV was less than 15 microns. When 1% porphyrin-lipid was incorporated into the bilayer, no membrane opening was observed, despite the much higher fluorescence observed due to an absence of self-quenching. Various GPV responses to laser irradiation were categorized as no response, membrane stretching, opening alone, or opening and closing (see FIG. 8 for an example of membrane stretching). None of the varying laser powers examined could induce opening in the highly fluorescent 1% porphyrin-lipid microvesicles (FIG. 2c). However, opening and closing was observed consistently for the 70 molar % GPVs, with the percentage of opening and closing events increasing with greater laser power. A small subset of GPVs remained open and did not reseal even after several minutes. A similar trend was observed as irradiation area was increased, effectively decreasing the laser fluence (FIG. 2d). Porphyrin fluorescence self-quenching has been shown to be highly correlated to self-quenching of singlet oxygen quantum yield.¹³ Although the less quenched, 1% pyro-lipid GPVs generated substantially more fluorescence and thus more singlet oxygen (hundreds of fold more, based on the quenching in porphysomes of similar composition¹¹), they did not open or close in response to laser irradiation. This is consistent with previous examination of singlet oxygen generation of porphyrins anchored in low molar percentage (1-10%) in phospholipid giant unilamellar vesicles (GUVs), which did not produce visible membrane poration in response to irradiation.¹⁴

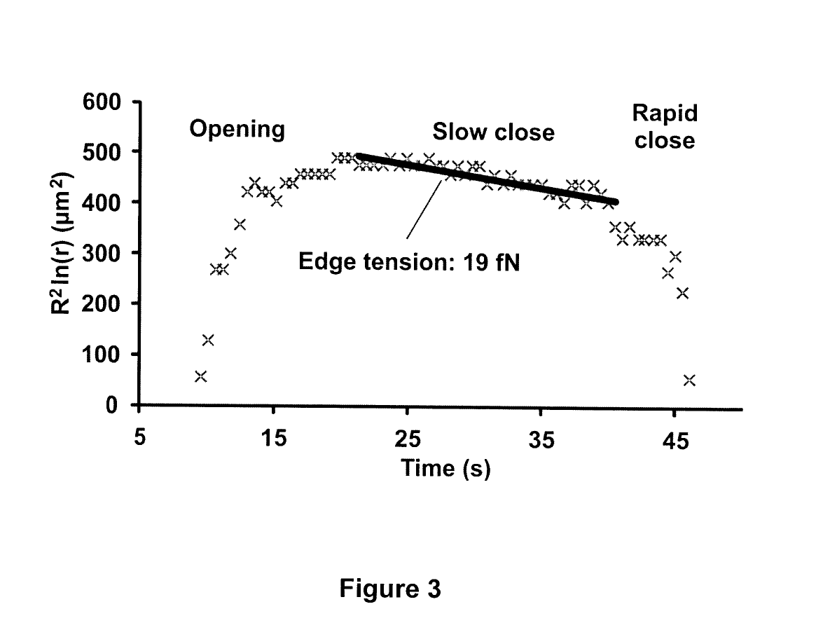

A substantial amount of experimental and theoretical work has led to an understanding of pressure-induced opening and closing of conventional GUVs.^{7,8,15,16} In these well-established models, pore opening is initiated and propagated by increased surface tension. Once the pore forms, lipids re-orient themselves to minimize hydrophobic side-chain exposure to the aqueous environment, but this modified packing structure has a free energy cost. Thus, an edge tension force is generated that opposes pore formation and is responsible for pore closure. Pore dynamics are balanced by the opposing forces of edge tension and surface tension. We hypothesized that in the case of GPVs, the porphyrin bilayer may stabilize the pore edge and reduce the edge tension force. As shown in FIG. 3, a typical GPV opening followed conventional patterns of GUV opening, with a rapid opening, slow closing and fast closing phase. Based on mathematical models developed by the Brochard-Wyart group and recently further elucidated by the Dimova group, it has been demonstrated that edge tension can be calculated during the slow closure period from the slope of R²In(r) as a function of time, where R is the radius of the GUV and r is the radius of the pore.^{8,15} The edge tension, γ, can be calculated from equation 1:

γ=−(3/2)πηa (1)

a represents the slope of the linear fit of the slow closure phase shown in FIG. 3 and η is the viscosity of the medium, water in this case. Using this technique, we estimate a typical edge tension force during GPV closing of 19 fN. This value is noteworthy as it is approximately 3 orders of magnitude smaller than conventional phospholipid bilayers.⁸ Thus, the edge of the porated porphyrin bilayer appears to be significantly stabilized by the porphyrin itself. Without being bound to any theory, this may be from the extensive and dynamic face to face porphyrin pi-pi electron interactions that occur in the GPV bilayer. Poration initiation and formation is likely due to some photophysical process that has an analogous effect to increasing in the membrane tension near the pore site. Two possible scenarios are that localized heating generates energy that can separate the GPV, or that localized bleaching of the bilayer creates a modified chemical species that causes the GPV opening until other porphyrin-lipid re-diffuses into position and the bilayer reseals.

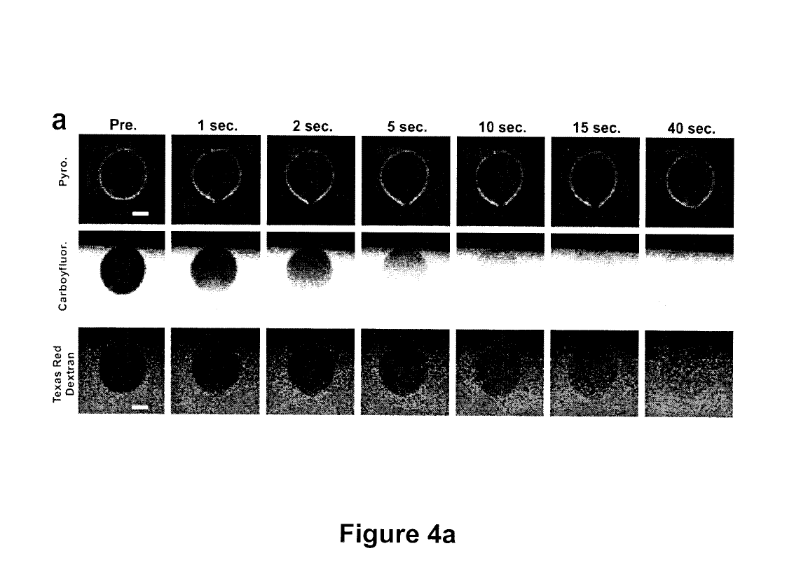

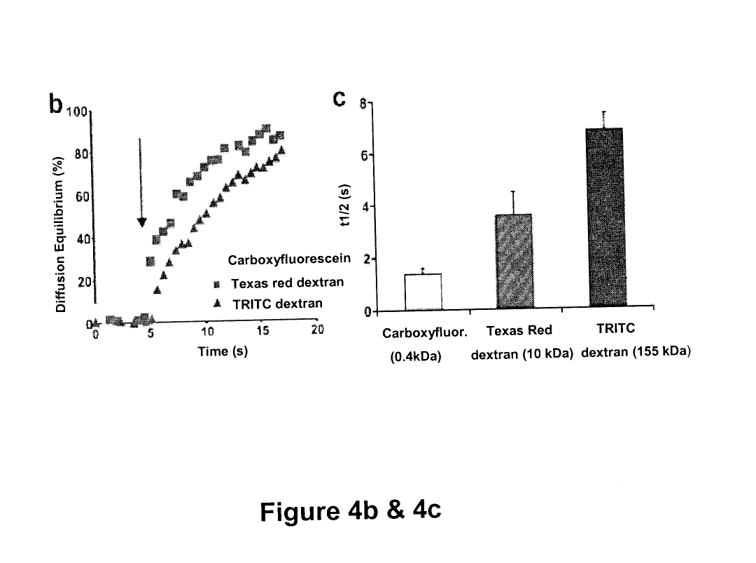

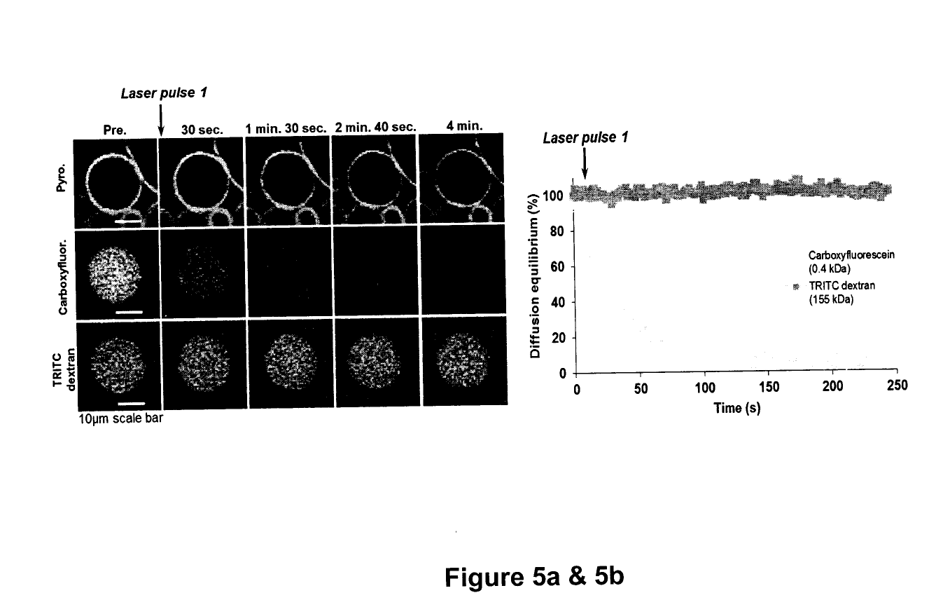

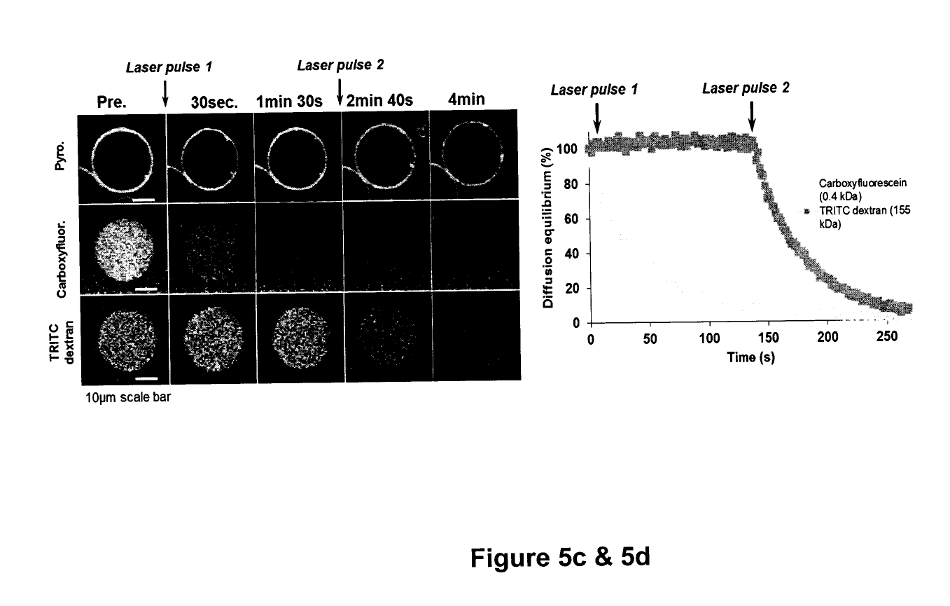

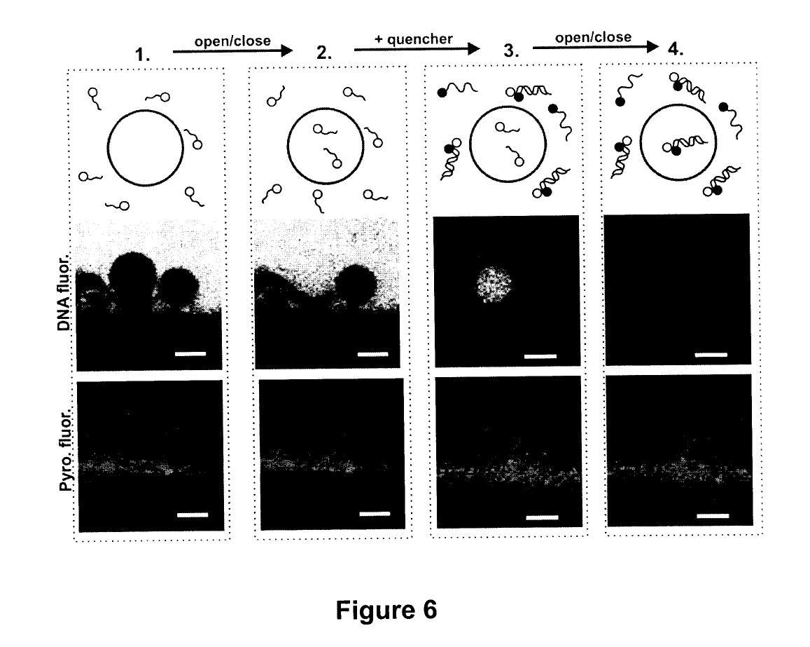

To verify the integrity of the porphyrin bilayer and to determine whether the opening process could control the passage of external molecules, we added two fluorophores to the solution outside the GPVs. One was carboxyfluorescein, a small molecule and the other was a large, Texas Red labeled dextran. No fluorescence was observed inside the GPVs following addition of the fluorophores into the solution, demonstrating that the porphyrin bilayer was impermeable to these molecules (FIG. 3a). Following laser irradiation, the GPV opened, and both fluorophores entered its interior. It appeared the smaller carboxyfluorescein fluorophores entered the GPV faster than the Texas Red dextran, as would be expected for smaller molecules, which diffuse more rapidly. We quantified the internalization rate of three fluorophores of varying sizes and observed that the smaller 0.4 kDa carboxyfluorescein diffused in faster than the 10 kDa dextran, which in turn diffused faster than the 150 kDa dextran (FIG. 4b). The time required for half the molecules to diffuse into the GPVs following opening varied from 1 to 8 seconds based on the size of the cargo (FIG. 4c). Using this technique, cargo of specific size could be selectively released (or loaded) into the GPV by varying the laser fluence (adjusting the irradiation spot diameter and irradiation time). Carboxyfluorescein (0.4 kDa) and TRITC-dextran (155 kDa) were encapsulated in the GPVs and exterior fluorophores were washed away. Using a laser fluence of 2 μJ/μm², carboxyfluorescein was released upon irradiation of the GPV membrane, however, TRITC-dextran remained encapsulated (FIGS. 5a and b). In another scenario, after applying a first laser pulse with low laser fluence (2 μJ/μ²), carboxyfluorescein was released. After 2 minutes, another laser pulse of higher laser fluence (20 μJ/μm²) was applied and the TRITC-dextran was released. This illustrates the concept of optical gating, allowing specific cargo in and out of the GPV in a size-dependent manner. To demonstrate that a GPV could be repeatedly optically manipulated, laser induced membrane-opening was used to control the passage of oligonucleotides (FIG. 6). When a fluorescein-labeled DNA oligonucleotide was incubated with GPVs, it remained outside the vesicles. Upon laser-induced opening, the DNA diffused into the GPV, as indicated by an increase in internalized fluorescence. Next, a complementary strand labeled with dabcyl, a dark quencher, was added to the external buffer. The fluorescence of the solution was markedly attenuated. However, the interior of the GPV remained fluorescent, as the quenching oligonucleotide could not pass the porphyrin bilayer to reach the DNA that had been passively transported there. Finally, when the GPV was opened again, the quencher and quenched hybridized DNA could diffuse into the interior and eliminate the fluorescence coming from the GPV.

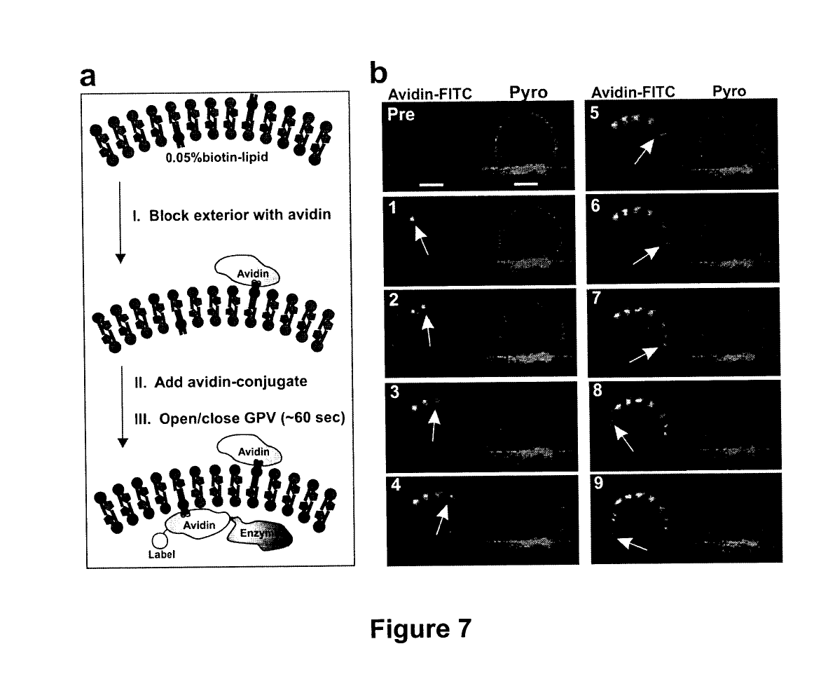

A useful enclosed microreactor should confine the desired reaction to the interior space of the vesicle. We developed a strategy to selectively attach enzymatic molecules of interest to the interior of GPVs (FIG. 7a). By including a small molar percentage of biotinylated lipid in the formulation, GPVs could be formed that were prone to avidin binding, which essentially an irreversibly association with biotin in standard aqueous conditions. The exterior of the GPVs were blocked with a 2 fold molar excess of avidin, ensuring all biotin sites on the exterior leaflet of the GPV bilayer were occupied. A four excess of fluorescein labeled avidin was then added to the external medium. The GPV was then opened using laser irradiation. The labeled avidin did not freely diffuse into the GPV and bind uniformly around the circumference. Instead, it bound exclusively around the site of membrane opening inside the GPV (FIG. 7b). This may be due to the smaller opening that was induced in the membrane when the biotin and avidin was used. This process was repeated 8 times to achieve uniform spacing of labeled avidin around the periphery of the GPV interior. In this case, we used fluorescently labeled avidin, but enzyme-avidin conjugates are also available and could be placed in the GPV interior in the same manner. Eventually, following selective enzyme attachment to the GPV interior, substrates could be diffused into the GPVs to become enzymatically transformed into products and then released on demand by porphyrin bilayer opening. This approach could prove useful for small volume reactions for enzyme activity optimization, screening approaches or sequential reactions. The ability to robustly open and close membranes has broad reaching consequences and porphyrin bilayers enable programmable vesicle opening and closing.

Although preferred embodiments of the invention have been described herein, it will be understood by those skilled in the art that variations may be made thereto without departing from the spirit of the invention or the scope of the appended claims. All references mentioned herein, including in the following reference list, are incorporated in their entirety by reference.

### REFERENCES

1. Kurihara, K. et al. Self-reproduction of supramolecular giant vesicles combined with the amplification of encapsulated DNA. Nature Chem. 3, 775-781 (2011).
2. Mansy, S. S. et al. Template-directed synthesis of a genetic polymer in a model protocell. Nature 454, 122-125 (2008).
3. Luisi, P. L., Walde, P. & Oberholzer, T. Lipid vesicles as possible intermediates in the origin of life. Curr. Opin. Colloid Interface Science 4, 33-39 (1999).
4. Neumann, E. & Rosenheck, K. Permeability changes induced by electric impulses in vesicular membranes. J. Membrane Biol. 10, 279-290 (1972).
5. Hanahan, D. Studies on transformation of Escherichia coli with plasmids. J. Mol. Biol. 166, 557-580 (1983).
6. Yang, P., Lipowsky, R. & Dimova, R. Nanoparticle Formation in Giant Vesicles: Synthesis in Biomimetic Compartments. Small 5, 2033-2037 (2009).
7. Sandre, O., Moreaux, L. & Brochard-Wyart, F. Dynamics of transient pores in stretched vesicles. P.N.A.S., U.S.A. 96, 10591-10596 (1999).
8. Portet, T. & Dimova, R. A New Method for Measuring Edge Tensions and Stability of Lipid Bilayers Effect of Membrane Composition. Biophys. J. 99, 3264-3273 (2010).
9. Kyrsting, A., Bendix, P. M., Stamou, D. G. & Oddershede, L. B. Heat Profiling of Three-Dimensionally Optically Trapped Gold Nanoparticles using Vesicle Cargo Release. Nano Lett. 11, 888-892 (2011).
10. Karlsson, M. et al. Electroinjection of Colloid Particles and Biopolymers into Single Unilamellar Liposomes and Cells for Bioanalytical Applications. Anal. Chem. 72, 5857-5862 (2000).
11. Lovell, J. F. et al. Porphysome nanovesicles generated by porphyrin bilayers for use as multimodal biophotonic contrast agents. Nature Mater. 10, 324-332 (2011).
12. Angelova, M. I., Soléau, S., Méléard, P., Faucon, F. & Bothorel, P. Preparation of giant vesicles by external AC electric fields. Kinetics and applications. Trends in Colloid and Interface Science VI 89, 127-131 (1992).
13. Lovell, J. F. et al. FRET Quenching of Photosensitizer Singlet Oxygen Generation. J. Phys. Chem. B 113, 3203-3211 (2009).
14. Riske, K. A. et al. Giant Vesicles under Oxidative Stress Induced by a Membrane-Anchored Photosensitizer. Biophys. J. 97, 1362-1370 (2009).
15. Brochard-Wyart, F., de Gennes, P. G. & Sandre, O. Transient pores in stretched vesicles: role of leak-out. Physica A: Statistical Mechanics and its Applications 278, 32-51 (2000).
16. Karatekin, E., Sandre, O. & Brochard-Wyart, F. Transient pores in vesicles. Polymer International 52, 486-493 (2003).
17. Christian, D. A. et al. Spotted vesicles, striped micelles and Janus assemblies induced by ligand binding. Nature Mater. 8, 843-849 (2009).

1. A vesicle comprising a bilayer comprising porphyrin-phospholipid conjugate, wherein the porphyrin-phospholipid conjugate comprises one porphyrin, porphyrin derivative or porphyrin analog covalently attached to a lipid side chain, preferably at the sn-1 or the sn-2 position, of one phospholipids, wherein the vesicle is 1-100 microns in diameter.

2. The vesicle of claim 1, wherein the vesicle is 10-50 microns in diameter.

3. The vesicle of claim 1, comprising between 15-100 molar % porphyrin-phospholipid conjugate.

4. The vesicle of claim 1, comprising between 20-90 molar % porphyrin-phospholipid conjugate.

5. The vesicle of claim 1, comprising between 30-80 molar % porphyrin-phospholipid conjugate.

6. The vesicle of claim 1, comprising between 40-75 molar % porphyrin-phospholipid conjugate.

7. The vesicle of claim 1, comprising between 50-70 molar % porphyrin-phospholipid conjugate.

8. The vesicle of claim 1, comprising between 60-70 molar % porphyrin-phospholipid conjugate.

9. The vesicle of claim 1, comprising between 65-70 molar % porphyrin-phospholipid conjugate.

10. The vesicle of claim 1, comprising about 70 molar % porphyrin-phospholipid conjugate.

11. The vesicle of claim 1 wherein the porphyrin, porphyrin derivative or porphyrin analog in the porphyrin-phospholipid conjugate is selected from the group consisting of hematoporphyrin, protoporphyrin, tetraphenylporphyrin, a pyropheophorbide, a bacteriochlorophyll, chlorophyll a, a benzoporphyrin derivative, a tetrahydroxyphenyl chlorin, a purpurin, a benzochlorin, a naphthochlorins, a verdin, a rhodin, a keto chlorin, an azachlorin, a bacteriochlorin, a tolyporphyrin, a benzobacteriochlorin, an expanded porphyrin and a porphyrin isomer.

12. The vesicle of claim 11, wherein the expanded porphyrin is a texaphyrin, a sapphyrin or a hexaphyrin and the porphyrin isomer is a porphycene, an inverted porphyrin, a phthalocyanine, or a naphthalocyanine.

13. The vesicle of claim 1 wherein the phospholipid in the porphyrin-phospholipid conjugate comprises phosphatidylcholine, phosphatidylethanoloamine, phosphatidylserine or phosphatidylinositol.

14. The vesicle of claim 13, wherein the phospholipid comprises an acyl side chain of 12 to 22 carbons.

15. The vesicle of claim 1 wherein the porphyrin in the porphyrin-phospholipid conjugate is pyropheophorbide-a acid.

16. The vesicle of claim 1 wherein the porphyrin in the porphyrin-phospholipid conjugate is a bacteriochlorophyll derivate.

17. The vesicle of claim 1 wherein the phospholipid in the porphyrin-phospholipid conjugate is 1-Palmitoyl-2-Hydroxy-sn-Glycero-3-Phosphocholine or 1-Stearoyl-2-Hydroxy-sn-Gycero-3-Phosphocholine.

18. The vesicle of claim 1 wherein the porphyrin-phospholipid conjugate is pyro-lipid.

19. The vesicle of claim 1 wherein the porphyrin-phospholipid conjugate is oxy-bacteriochlorophyll-lipid.

20. The vesicle of claim 1 wherein the porphyrin is conjugated to the glycerol group on the phospholipid by a carbon chain linker of 0 to 20 carbons.

21. The vesicle of claim 1, wherein the vesicle is substantially spherical.

22. The vesicle of claim 1, having an enzyme attached to the inner surface of the bilayer.

23. The vesicle of claim 1, wherein the remainder of the bilayer is comprised substantially of other phospholipid.

24. The vesicle of claim 23, wherein the other phospholipid is selected from the group consisting of selected from the group consisting of phosphatidylcholines, phosphatidylethanolamines, phosphatidic acid, phosphatidylglycerols and combinations thereof.

25. The vesicle of claim 23, wherein the other phospholipid is selected from the group consisting of 1,2-dipalmitoyl-sn-glycero-3-phosphatidic acid (DPPA), 1,2-dipalmitoyl-sn-glycero-3-phosphatidylcholine (DPPC), 1,2-distearoyl-sn-glycero-3-phosphocholine (DSPC), 1,2-dimyristoyl-sn-glycero-3-phosphocholine (DMPC), 1,2-dibehenoyl-sn-glycero-3-phosphocholine (DBPC), 1,2-diarachidoyl-sn-glycero-3-phosphatidylcholine (DAPC), 1,2-dilignoceroyl-sn-glycero-3-phosphatidylcholine (DLgPC), 1,2-dipalmitoyl-sn-glycero-3-[phosphor-rac-(1-glycerol)] (DPPG), L-α-phosphatidylcholine, and combinations thereof.

26. The vesicle of claim 23 further comprising cholesterol.

27. The vesicle of claim 26 wherein the cholesterol is present in a molar ratio of 3:2 of remainder other phospholipid to cholesterol.

28. A method of preparing vesicles, comprising:
a. preparing a solution comprising porphyrin-phospholipid conjugate, wherein the porphyrin-phospholipid conjugate comprises one porphyrin, porphyrin derivative or porphyrin analog covalently attached to a lipid side chain of one phospholipid, preferably at the sn-1 or the sn-2 position; the solution optionally further comprising phospholipids and cholesterol;
b. dehydrating and rehydrating the solution and subjecting a resulting lipid film to an alternating current.

29. The method of claim 28, wherein the solution is coated onto wires, preferably platinum wires, which deliver the alternating current.

30. The method of claim 28, wherein the solution comprises chloroform as the solvent.

31. The method of claim 28, wherein the alternating current is controlled by an Arduino microcontroller.

32. The method of claim 31, wherein the Arduino microcontroller is a part of a circuit as described in FIG. 1a or 1c.

33. The method of claim 28 for preparing the vesicle of any one of claims 1-26.

34. A vesicle produced by the method of claim 28.

35. The vesicle of claim 1 produced by the method of claim 28.

36. A method of controlled opening of a vesicle, comprising providing the vesicle of claim 1 and irradiating the vesicle with a laser or other light source, preferably a xenon or halogen lamp, capable of opening the vesicle.

37. The method of claim 36, wherein the controlled opening is at a predetermined location on the vesicle bilayer and said location is irradiated with the laser.

38. The method of claim 36, wherein the controlled opening is at a predetermined time.

39. The method of claim 36, wherein the controlled opening is performed under a microscope.

40. The method of claim 36, wherein the laser power is about 660 μW.

41. The method of claim 40, wherein the laser has a wavelength of 405 nm.

42. The method of claim 36, wherein the vesicle is in a solution having a salt concentration of less than 4 mM.

43. The method of claim 36, wherein a size of the opening is controlled proportionally with the level of laser fluence.

44. (canceled)

45. A method of performing a bioreaction between at least two reagents in a vesicle, comprising,
a. providing the vesicle of claim 1 having a first reagent encapsulated therein;
b. performing controlled opening of the vesicle according to the method of claim 36 to allow the entry of a second reagent into the interior of the vesicle and optionally allowing the vesicle to self-close; and
c. allowing the bioreaction to occur.
